(19) United States
(12) Patent Application Publication
(10) Pub. No.: US 2011/0275564 A1
(43) Pub. Date: Nov. 10, 2011

(54) ERYTHROPOIETIN

(75) Inventors: Peter Artymiuk, Sheffield (GB); Richard Ross, Sheffield (GB); Jon Sayers, Sheffield (GB)

(73) Assignee: ASTERION LIMITED, Sheffield (GB)

(21) Appl. No.: 12/671,998

(22) PCT Filed: Aug. 4, 2008
(86) PCT No.: PCT/GB2008/002640

§ 371 Date: Jul. 27, 2011

Related U.S. Application Data
(60) Provisional application No. 60/956,319, filed on Aug. 16, 2007.

(30) Foreign Application Priority Data
Aug. 3, 2007 (GB) 0715126.9
May 21, 2008 (GB) 0809208.2

(51) Int. Cl. A61K 38/18; C07H 21/04; C07K 14/00; C12N 5/10; C12N 1/00; C12N 1/15; C12N 1/19; C07K 14/505; A61P 7/06

(52) U.S. Cl. 514/7.7; 530/380; 536/23.1; 530/350; 435/320.1; 435/243; 435/254.11; 435/254.21; 435/254.23; 435/325; 435/358; 435/365; 435/348; 435/419

(57) ABSTRACT

This disclosure relates to erythropoietin (EPO) fusion polypeptides; nucleic acid molecules encoding said polypeptides and methods of treatment that use said polypeptides.

The invention relates to erythropoietin (EPO) fusion polypeptides and dimers; nucleic acid molecules encoding said polypeptides and methods of treatment that use said polypeptides/dimers.

Cytokine receptors can be divided into two separate classes. Class 1 (referred to as the haematopoietic or growth hormone family) receptors are characterised by four conserved cysteine residues in the amino terminal part of their extracellular domain and the presence of a conserved Trp-Ser-Xaa-Trp-Ser motif in the C-terminal part. The receptors consist of two polypeptide chains. Class I receptors can be sub-divided into the GM-CSF sub-family (which includes IL-3, IL-5, GM-CSF, GCSF) and IL-6 sub-family (which includes IL-6, IL-11 and IL-12). In the IL-6 sub-family there is a common transducing subunit (gp130) that associates with one or two different cytokine subunits. There is a further sub-family referred to as the IL-2 sub-family (includes IL-2, IL-4, IL-7, IL-9 and IL-15. The repeated Cys motif is also present in Class 2 (interferon receptor family) the ligands of which are α, β and γ interferon but lack the conserved Trp-Ser-Xaa-Trp-Ser motif.

Human EPO is a 35 kD glycoprotein hormone involved in regulating red blood cell production in bone marrow. It is produced by the kidneys and since its purification in 1977 and cloning in 1985 it is used to treat anaemia in chronic kidney failure and other diseases. Cells that respond to EPO include endothelial cells, neural and cardiac cells. Preparations of EPO are heterogeneous due to differential glycosylation. The glycosylation of EPO is not required for in vitro activity indicating that the mature form of EPO can bind EPO receptor in the absence of glycosylation. However, the in vivo activity of EPO is dependent on the addition of carbohydrate moieties to prevent its degradation and delay clearance.

EPO activates red blood cell development through activation of a single high affinity receptor which is expressed at low levels on erthyroid cells and is absent in mature red blood cells. The EPO receptor (EPOR) belongs to the class I cytokine receptor super family. The binding of EPO to EPOR results in receptor dimerization which induces tyrosine phosphorylation of a number of cytoplasmic and membrane associated molecules, including EPOR. EPOR has been detected in myocytes, neuronal cells and endothelial cells and EPO is believed to act in controlling cardiac and brain development.

EPO has also been shown to protect the heart and brain against inflammatory and ischemic damage. The serum levels of EPO can be variable in response to normal and pathological conditions. The levels of EPO can increase by 100-1000 fold in response to hypoxia or blood loss.

Anaemia can result from a number pathological conditions. For example, an anaemic state can result from blood loss, haemolysis, iron deficiency, aplastic bone marrow or nutritional deficiencies. In some pathological conditions EPO levels can be much lower and results from chronic kidney failure which results in loss of production of EPO and consequent anaemia. In addition EPO levels can be low in patients suffering from diseases such as rheumatoid arthritis, AIDS and cancer (as a result of chemotherapy). Treatment of anaemia is very effective when recombinant EPO is administered (e.g. Epogen, Aransesp, and Epogin). Typically, recombinant EPO is administered twice or thrice weekly by subcutaneous injection or intravenously. The administration of EPO is not without risk. Some of the side effects include increased blood pressure, nausea, vomiting, headache and shortness of breath.

There is a need to develop additional forms of EPO that can be administered less frequently and has increased serum stability which results in the administration of lower dosages and fewer side effects.

This disclosure relates to the identification of EPO recombinant forms that have improved pharmacokinetics and activity. The new EPO molecules are biologically active, form dimers and have improved stability.

According to an aspect of the invention there is provided a nucleic acid molecule comprising a nucleic acid sequence that encodes a polypeptide that has the activity of erythropoietin wherein said polypeptide comprises erythropoietin, or part thereof linked, directly or indirectly, to the erythropoietin binding domain of the erythropoietin receptor.

According to an aspect of the invention there is provided a fusion polypeptide comprising: the amino acid sequence of erythropoietin, or active binding part thereof, linked, directly or indirectly, to the erythropoietin binding domain of the erythropoietin receptor.

In a preferred embodiment of the invention erythropoietin is linked to the binding domain of the of the erythropoietin receptor by a peptide linker; preferably a flexible peptide linker.

In a preferred embodiment of the invention said peptide linking molecule comprises at least one copy of the peptide Gly Gly Gly Gly Ser.

In a preferred embodiment of the invention said peptide linking molecule comprises 2, 3, 4, 5 or 6 copies of the peptide Gly Gly Gly Gly Ser.

Preferably said peptide linking molecule consists of 3 copies of the peptide Gly Gly Gly Gly Ser.

In an alternative preferred embodiment of the invention said peptide linking molecule consists of 4 copies of the peptide Gly Gly Gly Gly Ser.

In a still further alternative embodiment of the invention said polypeptide does not comprise a peptide linking molecule and is a direct fusion of erythropoietin and the erythropoietin binding domain of the erythropoietin receptor.

According to a further aspect of the invention there is provided a nucleic acid molecule comprising a nucleic acid sequence selected from:
i) a nucleic acid sequence as represented in SEQ ID NO:1;
ii) a nucleic acid sequence as represented in SEQ ID NO:3;
iii) a nucleic acid sequence as represented in SEQ ID NO: 5;
iv) a nucleic acid sequence as represented in SEQ ID NO:7; or
v) a nucleic acid molecule comprising a nucleic sequence that hybridizes under stringent hybridization conditions to SEQ ID NO:1, 3, 5 or 7 and which encodes a polypeptide that has erythropoietin receptor modulating activity.

In a preferred embodiment of the invention said polypeptide is an agonist.

In an alternative preferred embodiment of the invention said polypeptide is an antagonist.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ, is the temperature at which 50% of a given strand of a nucleic acid molecule is hybridized to its complementary strand. The following is an exemplary set of hybridization conditions and is not limiting:

Very High Stringency (allows sequences that share at least 90% identity to hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (allows sequences that share at least 80% identity to hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (allows sequences that share at least 50% identity to hybridize)
Hybridization: 6×SSC at RT to 55° C. for 16-20 hours
Wash at least twice: 2×-3×SSC at RT to 55° C. for 20-30 minutes each.

In a preferred embodiment of the invention said nucleic acid molecule comprises or consists of a nucleic acid sequence as represented in SEQ ID NO: 1.

In a preferred embodiment of the invention said nucleic acid molecule comprises or consists of a nucleic acid sequence as represented in SEQ ID NO: 3.

In a preferred embodiment of the invention said nucleic acid molecule comprises or consists of a nucleic acid sequence as represented in SEQ ID NO: 5.

In a preferred embodiment of the invention said nucleic acid molecule comprises or consists of a nucleic acid sequence as represented in SEQ ID NO: 7.

According to an aspect of the invention there is provided a polypeptide encoded by the nucleic acid according to the invention.

According to a further aspect of the invention there is provided a polypeptide comprising an amino acid sequence selected from:
i) an amino acid sequence as represented in SEQ ID NO:2;
ii) an amino acid sequence as represented in SEQ ID NO:4;
iii) an amino acid sequence as represented in SEQ ID NO:6;
iv) an amino acid sequence as represented in SEQ ID NO:8;
v) an amino acid sequence as represented in SEQ ID NO:17;
vi) an amino acid sequence as represented in SEQ ID NO:18; or
   wherein said polypeptide has erythropoietin receptor modulating activity.

In a preferred embodiment of the invention said polypeptide is an agonist.

In an alternative preferred embodiment of the invention said polypeptide is an antagonist.

In a preferred embodiment of the invention said polypeptide comprises or consists of an amino acid sequence as represented in SEQ ID NO: 2.

In a preferred embodiment of the invention said polypeptide comprises or consists of an amino acid sequence as represented in SEQ ID NO: 4.

In a preferred embodiment of the invention said polypeptide comprises or consists of an amino acid sequence as represented in SEQ ID NO: 6.

In a preferred embodiment of the Invention said polypeptide comprises or consists of an amino acid sequence as represented in SEQ ID NO: 8.

In a preferred embodiment of the invention said polypeptide comprises or consists of an amino acid sequence as represented in SEQ ID NO: 17.

In a preferred embodiment of the invention said polypeptide comprises or consists of an amino acid sequence as represented in SEQ ID NO: 18.

According to an aspect of the invention there is provided a homodimer consisting of two polypeptides wherein each of said polypeptides comprises:
i) a first part comprising erythropoietin, or a receptor binding domain thereof, optionally linked by a peptide linking molecule to
ii) a second part comprising an erythropoietin binding domain of the erythropoietin receptor.

In a preferred embodiment of the invention said homodimer comprises two polypeptides comprising or consisting of SEQ ID NO: 2.

In a preferred embodiment of the invention said homodimer comprises two polypeptides comprising or consisting of SEQ ID NO: 4.

In a preferred embodiment of the invention said homodimer comprises two polypeptides comprising or consisting of SEQ ID NO: 6.

In a preferred embodiment of the invention said homodimer comprises two polypeptides comprising or consisting of SEQ ID NO: 8.

In a preferred embodiment of the invention said homodimer comprises two polypeptides comprising or consisting of SEQ ID NO: 17.

In a preferred embodiment of the invention said homodimer comprises two polypeptides comprising or consisting of SEQ ID NO: 18.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a method to treat a human subject suffering from a condition that would benefit from administration of an erythropoietin agonist comprising administering an effective amount of at least one polypeptide according to the invention.

In a preferred method of the invention said polypeptide is administered intravenously.

In an alternative preferred method of the invention said polypeptide is administered subcutaneously.

In a further preferred method of the invention said polypeptide is administered at two day intervals; preferably said polypeptide is administered at weekly, 2 weekly or monthly intervals.

In a preferred method of the invention said condition is anaemia.

In a preferred method of the invention said condition is anaemia as a consequence of chronic kidney disease.

In a preferred method of the invention said condition is anaemia as a consequence of chemotherapy in the treatment of cancer.

In a preferred method of the invention said condition is anaemia as a consequence of chemotherapy in the treatment of rheumatoid arthritis.

In a preferred method of the invention said condition is anaemia as a consequence of chemotherapy in the treatment of AIDS.

According to an aspect of the invention there is provided the use of a polypeptide according to the invention for the manufacture of a medicament for the treatment of anaemia.

In a further preferred embodiment of the invention said polypeptide is administered at two day intervals; preferably said polypeptide is administered at weekly, 2 weekly or monthly intervals.

In a preferred method of the invention said condition is anaemia as a consequence of chronic kidney disease.

In an alternative preferred embodiment of the invention said condition is anaemia as a consequence of chemotherapy in the treatment of cancer.

In an alternative preferred embodiment of the invention said condition is anaemia as a consequence of chemotherapy in the treatment of rheumatoid arthritis.

In an alternative preferred embodiment of the invention said condition is anaemia as a consequence of chemotherapy in the treatment of AIDS.

According to a further aspect of the invention there is provided a monoclonal antibody that binds the polypeptide or dimer according to the invention.

Preferably said monoclonal antibody is an antibody that binds the polypeptide or dimer but does not specifically bind erythropoietin or erythropoietin receptor individually.

The monoclonal antibody binds a conformational antigen presented either by the polypeptide of the invention or a dimer comprising the polypeptide of the invention.

In a further aspect of the invention there is provided a method for preparing a hybridoma cell-line producing monoclonal antibodies according to the invention comprising the steps of:
i) immunising an immunocompetent mammal with an immunogen comprising at least one polypeptide according to the invention;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a hybridoma cell-line obtained or obtainable by the method according to the invention.

According to a further aspect of the invention there is provided a diagnostic test to detect a polypeptide according to the invention in a biological sample comprising:
i) providing an isolated sample to be tested;
ii) contacting said sample with a ligand that binds the polypeptide according to the invention; and
iii) detecting the binding of said ligand in said sample.

In a preferred embodiment of the invention said ligand is an antibody; preferably a monoclonal antibody.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

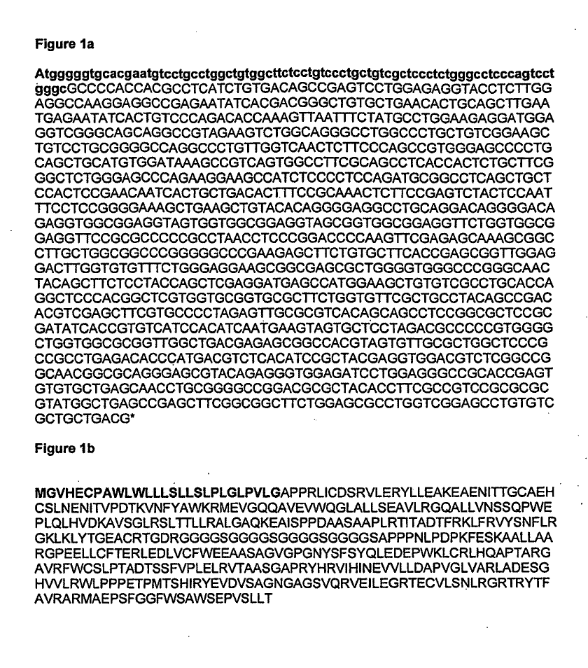

FIG. 1a is the nucleic acid sequence that encodes EPO-L1-EPOrEC: (EPO linked via a (G4S)4 to EPOrEC) for expression in mammalian cells (nucleotide sequence: length=1299 bp including signal sequence). The sequence contains the signal sequence in bold and lower case: *refers to stop codon; FIG. 1b amino acid sequence (length=406aa without signal sequence). Signal sequence shown in bold;

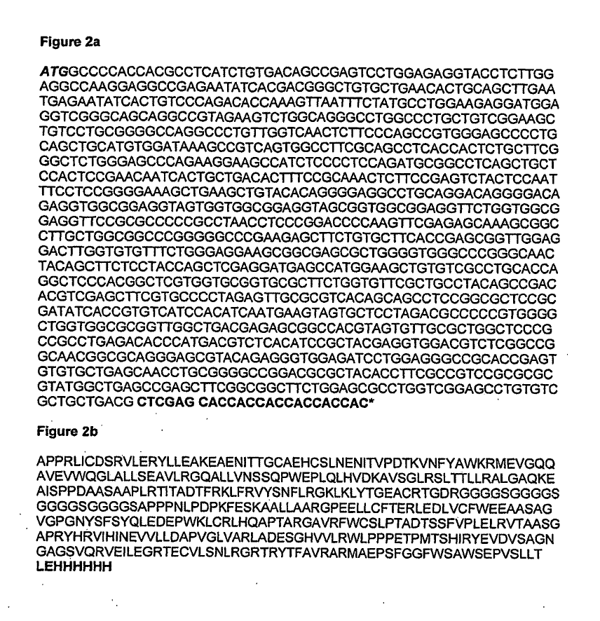

FIG. 2a is the nucleic acid sequence encoding EPO-L1-EPOrEC (EPO linked via a (G4S)4 to EPOrEC) for expression in bacterial cells, (nucleotide sequence: length=1242 bp). Bold letters refer to Xho1 and 6× Hist residues *refers to stop codon, ATG start codon shown in bold italics; FIG. 2b amino acid sequence (length=414aa);

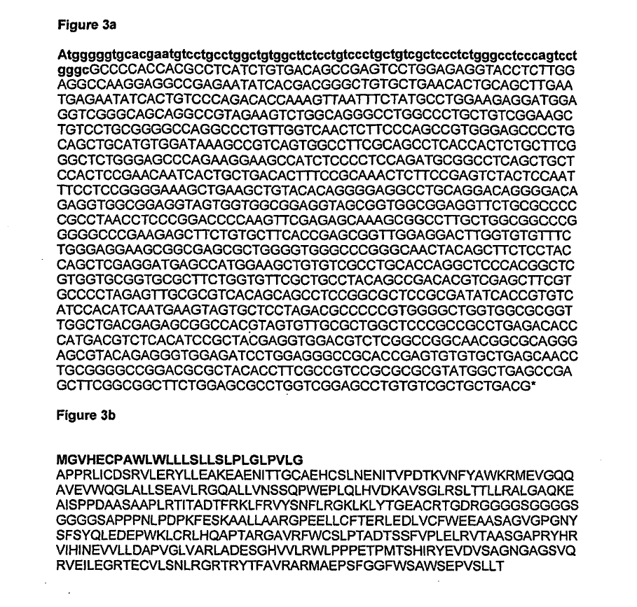

FIG. 3a is the nucleic acid sequence encoding EPO-L2-EPOrEC (EPO linked via a (G4S)3 to EPOrEC) for expression in mammalian cells (nucleotide sequence: length=1284 bp including signal sequence). Nucleotide and protein sequence represents the full sequence expressed in a mammalian cell line along with the signal sequence. Sequence contains the signal sequence in bold and lower case. *refers to stop codon; FIG. 3b amino acid sequence (length=401aa without signal sequence). Signal sequence shown in bold;

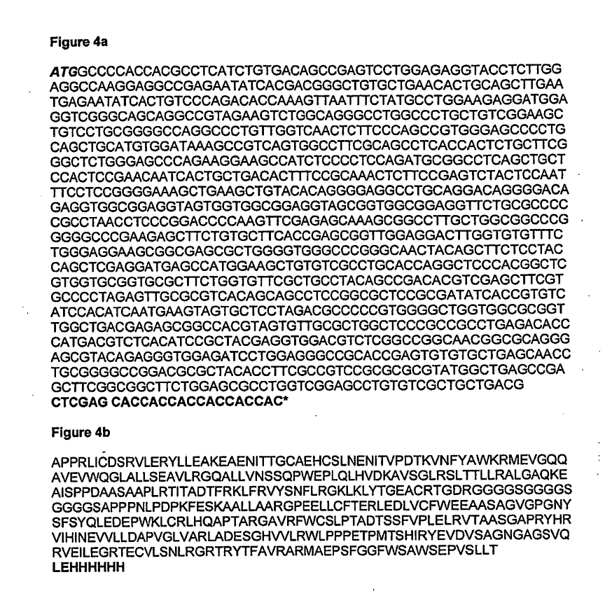

FIG. 4a is the nucleic acid sequence encoding EPO-L2-EPOrEC (EPO linked via a (G4S)3 to EPOrEC) for expression in bacterial cells (nucleotide sequence: length=1227 bp). Nucleotide and protein sequence represents the full sequence expressed in E. coli with a 6× Histidine tag and no signal sequence. Bold letters refer to Xho1 and 6× Hist residues. * refers to stop codon. ATG start codon shown in bold italics FIG. 4b amino acid sequence (length=409aa);

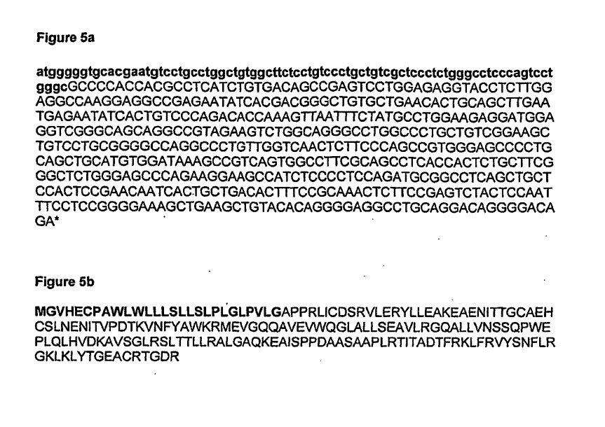

FIG. 5a is the nucleic acid sequence encoding EPO expressed in mammalian cells (nucleotide sequence: length=579 bp including signal sequence). Nucleotide and protein sequence represents the full sequence expressed in a mammalian cell line along with the signal sequence. Sequence contains the signal sequence in bold and lower case. *refers to stop codon; FIG. 5b amino acid sequence (length=166aa without signal sequence). Signal sequence shown in bold.

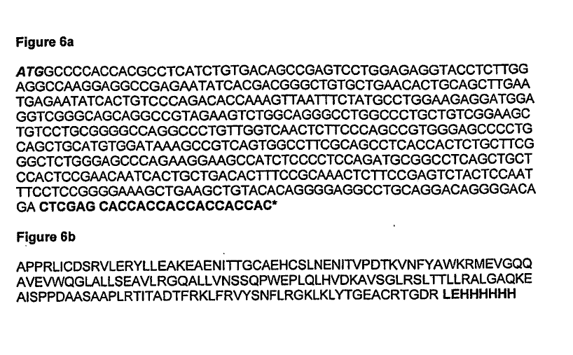

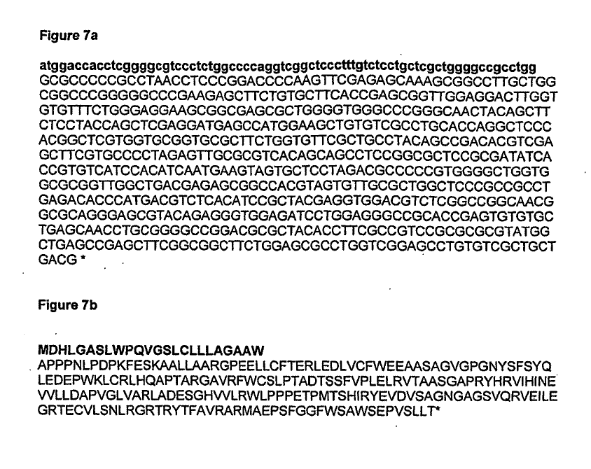

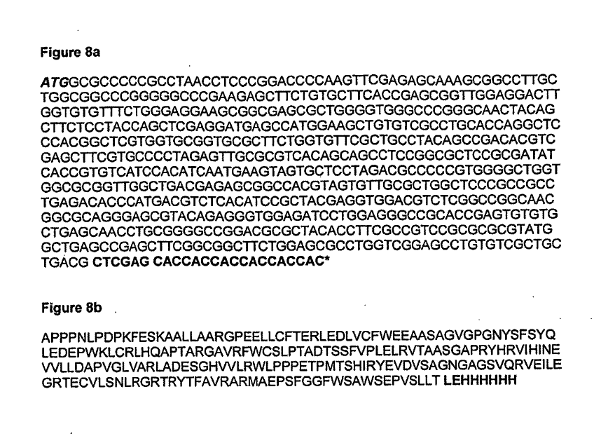

FIG. 6a is the nucleic acid sequence encoding EPO for expression in bacterial cells (nucleotide sequence: length=522 bp). Nucleotide and protein sequence represents the full sequence expressed in E. coli with a 6× Histidine tag. Bold letters refer to Xho1 and 6× Hist residues. *refers to stop codon. ATG start codon shown in bold italics; FIG. 6b amino acid sequence (length=174aa);

FIG. 7a is the nucleic acid sequence encoding EPOrEC expressed in mammalian cells (nucleotide sequence: length=732 bp including signal sequence). Nucleotide and protein sequence represents the full sequence expressed in mammalian cells along with the signal sequence; FIG. 7b amino acid sequence (length=220aa without signal sequence). Signal sequence is shown in bold;

FIG. 8a is the nucleic acid sequence encoding EPOrEC expressed in bacterial cells (nucleotide sequence: length=684 bp). Nucleotide and protein sequence represents the full sequence expressed in E. coli with a 6× Histidine tag. Bold letters refer to Xho1 and 6× Hist residues. *refers to stop codon. ATG start codon shown in bold italics; FIG. 8b amino acid sequence: (length=228aa);

FIG. 9 a) PCR was used to generate DNA consisting of the gene of interest flanked by suitable restriction sites (contained within primers R1-4). b) The PCR products were ligated into a suitable vector either side of the linker region. c) The construct was then modified to introduce the correct linker, which did not contain any unwanted sequence (i.e. the non-native restriction sites);

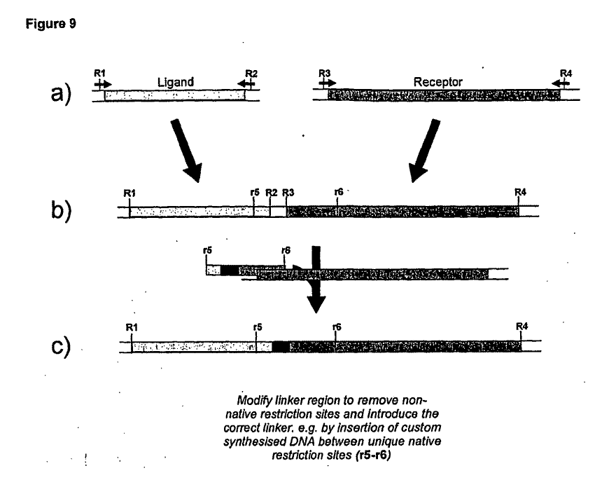

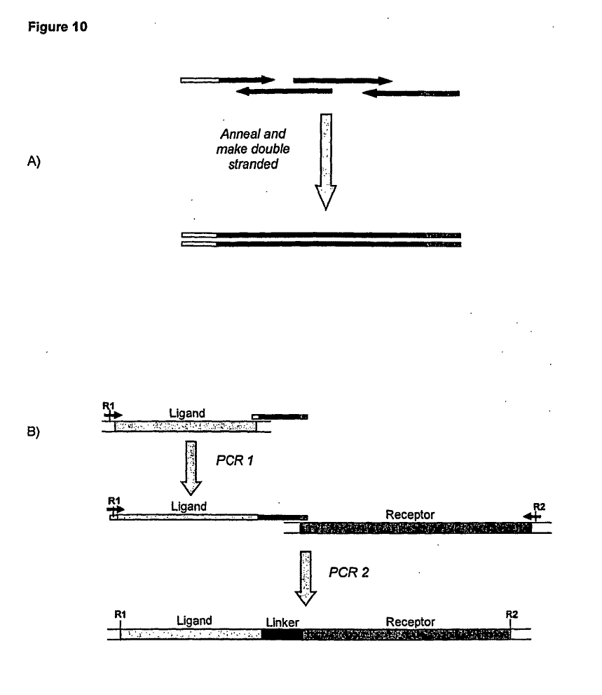

FIG. 10 a) Oligonucleotides were designed to form partially double-stranded regions with unique overlaps and, when annealed and processed would encode the linker with flanking regions which would anneal to the ligand and receptor. b) PCRs were performed using the “megaprimer” and terminal primers (R1 and R2) to produce the LR-fusion gene. The R1 and R2 primers were designed so as to introduce useful flanking restriction sites for ligation into the target vector;

FIG. 11 illustrates a western blot analysis of EPO fusion proteins. Western blot analysis of CHO flpIn stable cell lines expressing EPO and EPO chimeric constructs Lane 1=3B2; Lane 2=3B2; Lane 3=EPO; Lane 4=3A1; Lane 5=Mock media. A=Non reducing conditions; B=reducing conditions;

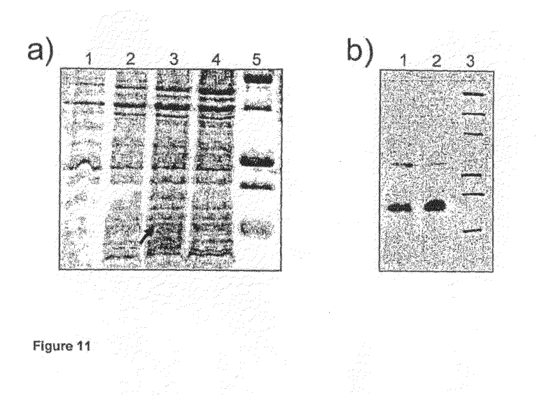

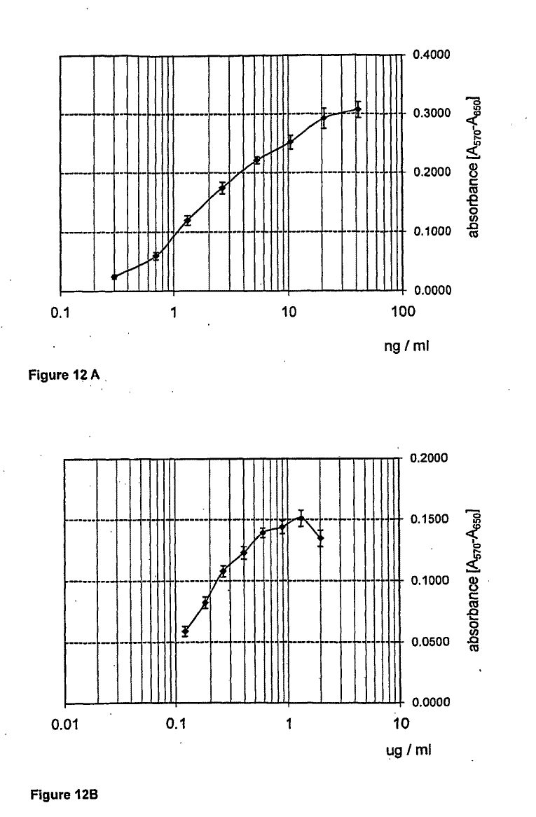

FIG. 12 illustrates the activity of an EPO fusion protein 3A1 compared to human EPO;

FIG. 12A shows the dose-response for EPO International Standard in the TF-1 cell proliferation assay; shows the dose-response for the AFT-EPO 3A1 in the TF-1 cell proliferation assay;

FIG. 13 is a graph showing the dose-response for EPO International Standard in the TF-1 cell proliferation assay;

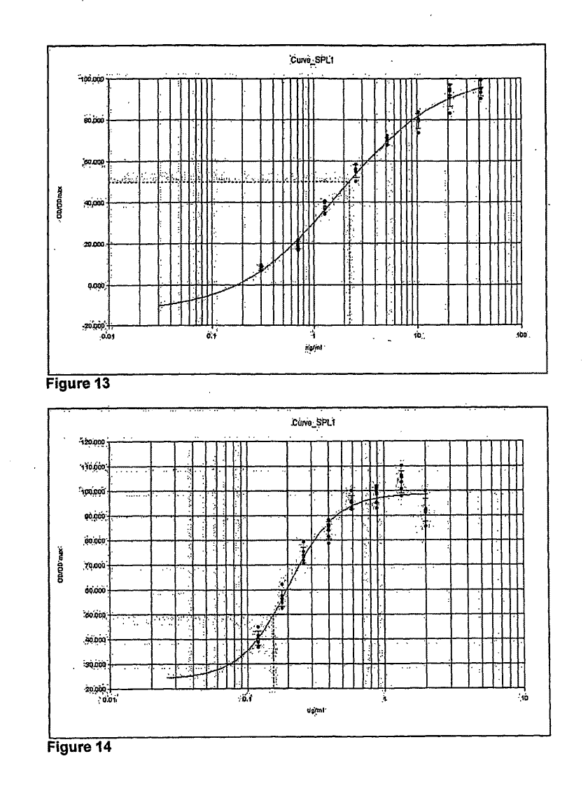

FIG. 14 is a graph showing the dose-response for the AFT-EPO fusion protein (3A1) in the TF-1 cell proliferation assay;

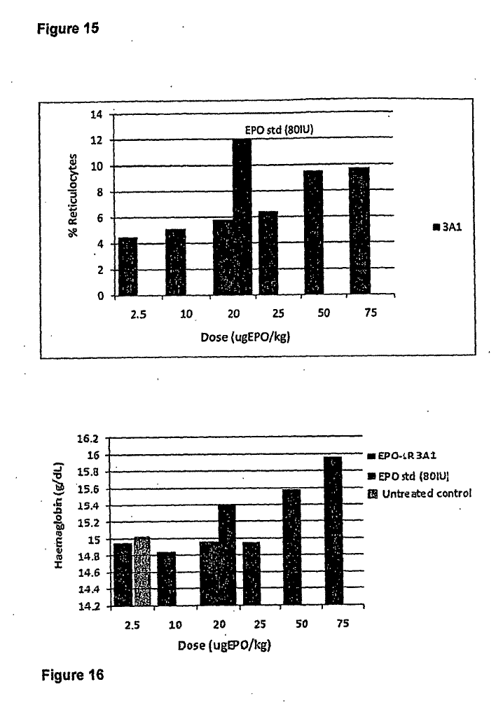

FIG. 15 is a graph showing the effect of 3A1 on the % retoculocytes compared to the untreated negative control and the EPO positive control;

FIG. 16 is a graph showing the effect of 3A1 on the haemaglobin count (g/dL) compared to the untreated negative control and the EPO positive control.

### MATERIALS AND METHODS

### In Vitro Testing

In vitro bioassays for epo are well known in the art. These measure epo induced stimulation of proliferation and/or differentiation of cells expressing EPOR. Some assays measure erythroid colony formation using bone marrow as a source of EPO responsive cells. Other bioassays measure proliferation using ³H-thymidine or differentiation by measuring incorporation of radioactive iron into haemoglobin in suspension cultures. The use of EPO responsive tumour cell lines (TF1 cells) or cells transformed with EPOR have been used for testing EPO agonist activity.

### In Vivo Testing

In vivo bioassays for erythropoietin are well known in the art and are based on the stimulation by exogenous EPO of red blood cell formation in an animal model, for example a rat, in which the erythroid progenitor pool has been expanded by hypoxia, bleeding or cytotoxic drugs. The response is typically measure by the incorporation of radioactive iron into spleen or red blood cells.

One such test is referred to as the exhypoxic polycythemia mouse assay and is an industrial standard for testing recombinant EPO (see Coles et al Nature 191: 1065, 1961).

### Immunological Testing

Immunoassays that measure the binding of EPO to polyclonal and monoclonal antibodies are known in the art. Commercially available EPO antibodies are available to detect EPO in samples and also for use in competitive inhibition studies. For example monoclonal antibodies can be purchased at http://www.ab-direct.com/index AbD Serotec.

### Recombinant Production of Fusion Proteins

The components of the fusion proteins were generated by PCR using primers designed to anneal to the ligand or receptor and to introduce suitable restriction sites for cloning into the target vector (FIG. 8a). The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector (FIG. 8b). The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification (FIG. 8c).

Alternatively, the linker with flanking sequence, designed to anneal to the ligand or receptor domains of choice, was initially synthesised by creating an oligonucleotide duplex and this processed to generate double-stranded DNA (FIG. 9a). PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice (FIG. 9b).

### Expression and Purification of Fusion Proteins

Expression was carried out in a suitable system (e.g. mammalian CHO cells, E. coli,) and this was dependant on the vector into which the EPO-fusion gene was generated. Expression was then analysed using a variety of methods which could include one or more of SDS-PAGE, Native PAGE, western blotting, ELISA well known in the art.

Stable transfected CHO Flp-In cell lines were grown in 75 cm2 flasks for approximately 3-4 days, at which point samples were taken for analysis. Samples were mixed with an equal volume of laemmli loading buffer in the presence and absence of 25 mM DTT and boiled for 5 minutes. Samples were separated on a 15% (w/v) bis-acrylamide gel and transferred to a PVDF membrane. After blocking in 5% (w/v) Milk protein in PBS-0.05% (v/v) Tween 20, sample detection was carried out using a specific anti-EPO antibody together with a Horse Radish Peroxidase (HRP) conjugated secondary antibody. Visualisation was by chemiluminesence on photographic film using an HRP detection kit; see FIG. 11.

Once a suitable level of expression was achieved the RL-fusions were expressed at a larger scale to produce enough protein for purification and subsequent analysis. Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin). Purified protein was analysed using a variety of methods which could include one or more of Bradford's assay, SDS-PAGE, Native PAGE, western blotting, ELISA.

### Characterisation of LR-Fusions

Denaturing PAGE, native PAGE gels and western blotting were used to analyse the fusion polypeptides and western blotting performed with antibodies non-conformationally sensitive to the LR-fusion. Native solution state molecular weight information can be obtained from techniques such as size exclusion chromatography using a Superose G200 analytical column and analytical ultracentrifugation.

### Statistics

Two groups were compared with a Student's test if their variance was normally distributed or by a Student-Satterthwaite's test if not normally distributed. Distribution was tested with an F test. One-way ANOVA was used to compare the means of 3 or more groups and if the level of significance was p<0.05 individual comparisons were performed with Dunnett's tests. All statistical tests were two-sided at the 5% level of significance and no imputation was made for missing values.

### In Vitro Bioaasay

### Cells Preparation

The bioactivity of 3A1 was determined in an in vitro TF-1 cell proliferation model. TF-1 cells (ATCC, Batch No. 5003310) were removed from liquid nitrogen storage and placed into a 37° C. waterbath for 2 min. The contents of the vial were then transferred to a 15 ml tube containing 9 ml of culture medium (10% FBS, 2 mM L-glutamine, 100 U/ml Penicillin, 100 μg/ml Streptomycin, 2 ng/ml GM-CSF in RPMI). Cells were centrifuged for 5 min at 123×g; the cell pellet was resuspended in culture medium and cell density adjusted to 4×10⁴ cells/ml.

### Cell Culture

Cells were cultured in CO₂ incubator (5% CO₂, 37° C.) In culture medium at a density of 2×10⁴−5×10⁴ cells/ml. Passages were performed twice a week ensuring cell density did not exceed 7×10⁵ cells/ml. Cell viability was assessed by trypan blue exclusion. Prior to assay cells were washed 3 times with PBS by spinning for 5 min at ˜125×g. The pellet was then reconstituted in assay medium (10% FBS, 100 U/ml Penicillin, 100 μg/ml Streptomycin, 2 mM L-glutamine in RPMI) and cell density was adjusted to 2×10⁵ cells/ml.

### Standards/Samples Preparation

EPO (international standard, NIBSC, Batch No 88/574) was reconstituted in assay medium to a concentration of 1 μg/ml (120 IU/ml), divided into 100 μl aliquiots and stored at −80° C. On each day of assay 1 vial was removed from the freezer and working concentrations were prepared.

### TF-1 Bioassay

50 μl of each protein tested was put into appropriate well in 96-wells microplate. Then 50 μl of cell suspension was added and the plate was shaken softly to allow cells and standard/samples to mix. Control wells contained only assay medium and cell suspension (50 μl+50 μl) and blank wells only assay medium (100 μl). Cells were exposed to different concentrations of test proteins for 72 hours in CO₂ incubator (5% CO₂, 37 C) and then 10 μl of MTT was added to each well. After 4 hours of incubation (5% CO₂, 37 C) solubilization buffer was added (100 μl/well) and the plate was left overnight in CO₂ incubator. Absorbance was then read at 570 nm and 650 nm (reference).

### Calculation of Results

All raw data were analysed using Gen5 software with the following transformations:
1. Results from reference (650 nm) were subtracted from the results obtained at 570 nm
2. Results from controls were then subtracted from the results obtained for cells exposed to different concentrations of test protein
3. Results obtained from step 2 were plotted as a ratio of OD/ODmax (%)

Based on the respective dose response curves (logarithmic, 4-parametrical) the values of EC₅₀ (the concentration which causes 50% of maximal response) for each protein were calculated.

### In Vivo Bioassay

EPO-LR fusion protein (3A1) was tested in the Normocythaemic mouse model as detailed below. The Normocythaemic mouse model is based on the measurement of stimulation of reticulocyte production in mice. Additionally the model can be used to detect biological activity of Erythropoietin (EPO) or EPO mimetic proteins thereof. Animal type: 32 B6D2F1/OlaHsd Mice, female, age 6-9 weeks at the commencement of the study. 4 mice were used for each treatment. The animals were derived from a controlled full barrier maintained breeding system (SPF) sourced from Harlan Winkelmann GmbH.

At the beginning of the assay procedure the mice were randomly distributed into 4 mice per cage. These animals were marked for identification by tail-painting (one label per cage). The animals were injected subcutaneously with 0.5 mL of the appropriate treatment (one solution per cage) and put into a new cage. The mice were combined in such a way that each cage housing the treated mice contained one mouse out of each different treatment. Four days after the injections, blood was collected from the tail vein and the number of reticulocytes was determined by flow cytometry.

### Example 1

The EPO gene was obtained as an IMAGE clone and subcloned into the expression vectors pET21a+(for E. coli expression) and pSecTag (for mammalian expression). PCR was used to produce DNA containing the EPO gene flanked by suitable restriction sites for insertion into the vectors.

For bacterial expression the primers used were NdeEPOF (5′-aattaattcat atggccccaccacgcctcatctg-3′) and EPDXhoR (5′-aattctcgagtctgtcccctgtcctgcag-3′) for Histidine-tagged protein and NdeEPOF and EPO*XhoR (5′-aattctcgagct atctgtcccctgtcctgcag-3′) for untagged protein. The PCR products were then ligated between NdeI and XhoI sites in pET21a+. The resulting clones were confirmed by sequencing.

Expression of EPO from E. coli was carried out in E. coli BL21 (DE3) RIPL cells which were transformed with the pET21a+EPO+/−His plasmids. Expression was induced with IPTG and confirmed by western blot using antibodies against EPO (FIG. 2X).

For mammalian expression the primers used were NheEPOF (5′-aaatttgctagccacc atgggggtgcacgaatgtcctg-3′) and EPO*H3R (5′-aattaagcttctatctgtcccctgtcctgcag-3′). The PCR product was then ligated between NheI and HindIII sites in pSecTag.

### Example 2

The EPOR gene was custom gene synthesised/obtained from a foetal liver cDNA library and subcloned into the expression vectors pET21a+(for E. coli expression) and pSecTag (for mammalian expression). PCR was used to produce DNA containing the EPO gene flanked by suitable restriction sites for insertion into the vectors.

For bacterial expression the primers used were NdeEPORFor (5′-gcgcataCATATGg cgcccccgcctaacctccc-3′) and EPORXhoRev2 (5′-gcgcCTCGAGCGTCAGCAGCGACAC AGGCT-3′) for Histidine-tagged protein and NdeEPOF and EPOR*XhoRev2 (5′-gcg cCTCGAGtcaCGTCAGCAGCGACACAGGCT-3′) for untagged protein. The PCR products were then ligated between NdeI and XhoI sites in pET21a+. The resulting clones were confirmed by sequencing.

For mammalian expression the primers used were NheEPORFor (5′-gcgcGCTAGCcacc atggaccacctcggggcgtc-3′) and EPORHindRev2 (5′-gcgcAAGCTTtcaCGTCAGCAGCG ACACAGGCT-3′). The PCR product was then ligated between NheI and HindIII sites in pSecTag.

### Example 3

The (G₄S)₃ linker with flanking sequence complementary to EPO at one end and EPOR at the other was synthesised by annealing three oligonucleotides; EPOlink3F (5′-agg tagtggtggcggaggtagcggtggcgg-3′), EPOlink3R1(5′-gggaggttaggcgggggcgcagaacctccgcc accgctacc-3′) and EPOlink3R2 (5′-tccgccaccactacctccgccacctctgtcccctgtcctgcag-3′), together to form a multiplex of oligonucleotides. This multiplex was then processed to create double-stranded DNA which could be used as a primer in a PCR.

PCRs were then performed using the primers BamNheEPOFor (5′-aaatttggatcc gctagccaccatgggggtgcacgaatgtcctg-3′), EPORHindRev2 (5′-gcgcAAGCTTtcaCGTC AGCAGCGACACAGGCT-3′) and the linker “megaprimer” produced previously with EPO and EPOR as the templates. This produced the EPO-(G₄S)₃-EPOrEC gene flanked by suitable restriction sites for ligation into the mammalian expression vector pSecTag (NheI and HindIII) and the phage vector M13mp18 (BamHI and HindIII).

For bacterial expression the EPO-(G₄S)₃-EPOrEC gene was generated with NdeI and *XhoI flanking regions by PCR using the primers NdeEPOF (5′-aattaattcata tggccccaccacgcctcatctg-3′) and EPOR*XhoRev2 (5′-gcgcCTCGAGtcaCGTCAGCAGCGACACAGGCT-3′). This was then ligated into pET21a+.

### Example 4

FIG. 11 illustrates the expression and detection by of two examples of EPO receptor fusion proteins; 3A1 and 3B2. Both 3A1 and 3B2 run between 75 and 100 kDa. EPO runs between 37 and 45 kDa as expected for the glycosylated protein.

The biological activity of 3A1 is assayed compared to human EPO and is illustrated in FIGS. 12, 13 and 14.

### Example 5

The dose-range examined for 3A1 was from 2.5 to 150 ug (mass equivalent to standard EPO) per kg of animal body weight. FIGS. 15 and 16 shows the in vivo activity of 3A1 against reticulocytes and haemoglobin following 4-days post dosing.

1. (canceled)

2. A fusion polypeptide comprising: the amino acid sequence of erythropoietin, or active binding part thereof, linked, directly or indirectly, to the erythropoietin binding domain of the erythropoietin receptor.

3. A fusion polypeptide according to claim 2 wherein erythropoietin is linked to the binding domain of the erythropoietin receptor by a peptide linker.

4. A fusion polypeptide according to claim 3 wherein said peptide linker comprises at least one copy of the peptide Gly Gly Gly Gly Ser (SEQ ID NO: 19).

5-7. (canceled)

8. A fusion polypeptide according to claim 2 wherein said polypeptide does not comprise a peptide linking molecule and is a direct fusion of erythropoietin and the erythropoietin binding domain of the erythropoietin receptor.

9. An isolated nucleic acid molecule comprising a nucleic acid sequence selected from:
i) a nucleic acid sequence as represented in SEQ ID NO:1;
ii) a nucleic acid sequence as represented in SEQ ID NO:3;
iii) a nucleic acid sequence as represented in SEQ ID NO: 5;
iv) a nucleic acid sequence as represented in SEQ ID NO:7; or
v) a nucleic acid sequence that hybridizes under stringent hybridization conditions to SEQ ID NO:1, 3, 5 or 7 and which encodes a polypeptide that has erythropoietin receptor modulating activity.

10-15. (canceled)

16. An isolated polypeptide encoded by the nucleic acid according to claim 9.

17. An isolated polypeptide comprising an amino acid sequence selected from:
i) an amino acid sequence as represented in SEQ ID NO:2;
ii) an amino acid sequence as represented in SEQ ID NO:4;
iii) an amino acid sequence as represented in SEQ ID NO:6;
iv) an amino acid sequence as represented in SEQ ID NO:8;
v) an amino acid sequence as represented in SEQ ID NO:17; or
vi) an amino acid sequence as represented in SEQ ID NO:18;
wherein said polypeptide has erythropoietin receptor modulating activity.

18-25. (canceled)

26. A homodimer consisting of two polypeptides wherein each of said polypeptides comprises:
i) a first part comprising erythropoietin, or a receptor binding domain thereof; and
ii) a second part comprising at least one erythropoietin binding domain of the erythropoietin receptor.

27. A homodimer according to claim 26 wherein said homodimer comprises two polypeptides comprising SEQ ID NO: 2, 4, 6, 8, 17 or 18.

28-32. (canceled)

33. A vector comprising a nucleic acid molecule according to claim 9.

34. A cell transfected or transformed with a nucleic acid molecule according to claim 9.

35-36. (canceled)

37. A pharmaceutical composition comprising a polypeptide according to claim 2 and an excipient or carrier.

38. (canceled)

39. A method to treat a human subject suffering from a condition that would benefit from administration of an erythropoietin agonist comprising administering an effective amount of at least one polypeptide according to claim 2.

40. A method according to claim 39 wherein said condition is anaemia.

41-59. (canceled)


SEQUENCE LISTING (36 sequences)

SEQ ID NO: 1 - DNA, 1299 bp, artificial
  EPO/EPOR fusion polypeptide
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggtggcggaggtagtggtggcggaggtagcggtggcggaggttctggtggcggaggttccgcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacg

SEQ ID NO: 2 - protein, 433 aa, Artificial
  EPO/EPOR fusion protein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSGGGGSGGGGSGGGGSAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLT

SEQ ID NO: 3 - DNA, 1245 bp, Artificial
  EPO/EPOR fusion protein
atggccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggtggcggaggtagtggtggcggaggtagcggtggcggaggttctggtggcggaggttccgcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgctcgagcaccaccaccaccaccac

SEQ ID NO: 4 - protein, 414 aa, Artificial
  EPO/EPOR fusion protein
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSGGGGSGGGGSGGGGSAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTLEHHHHHH

SEQ ID NO: 5 - DNA, 1284 bp, Artificial
  EPO/EPOR fusion protein
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggtggcggaggtagtggtggcggaggtagcggtggcggaggttctgcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacg

SEQ ID NO: 6 - protein, 428 aa, Artificial
  EPO/EPOR fusion protein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSGGGGSGGGGSAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLT

SEQ ID NO: 7 - DNA, 1230 bp, Artificial
  EPO/EPOR fusion protein
atggccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggtggcggaggtagtggtggcggaggtagcggtggcggaggttctgcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgctcgagcaccaccaccaccaccac

SEQ ID NO: 8 - protein, 409 aa, Artificial
  EPO/EPOR fusion protein
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSGGGGSGGGGSAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTLEHHHHHH

SEQ ID NO: 9 - DNA, 579 bp, Homo sapiens
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacaga

SEQ ID NO: 10 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 11 - DNA, 525 bp, artificial
  EPO engineered for bacterial expression
atggccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagactcgagcaccaccaccaccaccac

SEQ ID NO: 12 - protein, 174 aa, artificial
  EPO engineered for expression in bacteria
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRLEHHHHHH

SEQ ID NO: 13 - DNA, 732 bp, Homo sapiens
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacg

SEQ ID NO: 14 - protein, 244 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLT

SEQ ID NO: 15 - DNA, 687 bp, artificial
  EPOrEC engineered for expression in bacteria
atggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgctcgagcaccaccaccaccaccac

SEQ ID NO: 16 - protein, 228 aa, artificial
  EPOrEC engineered for bacterial expression
APPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTLEHHHHHH

SEQ ID NO: 17 - protein, 406 aa, Artificial
  EPO/EPOR fusion protein
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSGGGGSGGGGSGGGGSAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLT

SEQ ID NO: 18 - protein, 401 aa, Artificial
  EPO/EPOR fusion protein
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSGGGGSGGGGSAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLT

SEQ ID NO: 19 - protein, 5 aa, artificial
  peptide linker
GGGGS

SEQ ID NO: 20 - DNA, 34 bp, Artificial
  EPO forward oligonucleotide primer
aattaattcatatggccccaccacgcctcatctg

SEQ ID NO: 21 - DNA, 29 bp, Artificial
  EPO reverse olionucleotide primer
aattctcgagtctgtcccctgtcctgcag

SEQ ID NO: 22 - DNA, 32 bp, Artificial
  alternative EPO reverse oligonucleotide primer
aattctcgagctatctgtcccctgtcctgcag

SEQ ID NO: 23 - DNA, 38 bp, Artificial
  alternative EPO forward oligonucleotide primer
aaatttgctagccaccatgggggtgcacgaatgtcctg

SEQ ID NO: 24 - DNA, 32 bp, Artificial
  alternative reverse EPO oligonucleotide primer
aattaagcttctatctgtcccctgtcctgcag

SEQ ID NO: 25 - DNA, 30 bp, Artificial
  linker oligonucleotide primer
aggtagtggtggcggaggtagcggtggcgg

SEQ ID NO: 26 - DNA, 41 bp, Artificial
  linker oligonucleotide primer 2
gggaggttaggcgggggcgcagaacctccgccaccgctacc

SEQ ID NO: 27 - DNA, 43 bp, artificial
  linker oligonucleotide primer 2
tccgccaccactacctccgccacctctgtcccctgtcctgcag

SEQ ID NO: 28 - DNA, 44 bp, Artificial
  forward EPO oligonucleotide primer
aaatttggatccgctagccaccatgggggtgcacgaatgtcctg

SEQ ID NO: 29 - DNA, 33 bp, Artificial
  reverse EPO oligonucleotide primer
gcgcaagctttcacgtcagcagcgacacaggct

SEQ ID NO: 30 - DNA, 34 bp, Artificial; identical to SEQ ID NO: 20
  forward EPO oligonucleotide primer

SEQ ID NO: 31 - DNA, 33 bp, Artificial
  reverse EPO oligonucleotide primer
gcgcctcgagtcacgtcagcagcgacacaggct

SEQ ID NO: 32 - DNA, 33 bp, Artificial
  EPO receptor oligonucleotide primer
gcgcatacatatggcgcccccgcctaacctccc

SEQ ID NO: 33 - DNA, 30 bp, Artificial
  EPO receptor oligonucleotide primer
gcgcctcgagcgtcagcagcgacacaggct

SEQ ID NO: 34 - DNA, 33 bp, Artificial; identical to SEQ ID NO: 31
  EPO receptor oligonucleotide primer

SEQ ID NO: 35 - DNA, 34 bp, Artificial
  EPO receptor oligonucleotide primer
gcgcgctagccaccatggaccacctcggggcgtc

SEQ ID NO: 36 - DNA, 33 bp, Artificial; identical to SEQ ID NO: 29
  EPO receptor oligonucleotide primer